(19) United States
(12) Patent Application Publication
(10) Pub. No.: US 2008/0005813 A1
(43) Pub. Date: Jan. 3, 2008

(54) Polypeptide Having Function of Cinnamyl Alcohol Dehydrogenase, Polynucleotide Encoding the Polypeptide and Those Uses

(75) Inventors: Dong-hee Lee, Busan (KR); Tae-Hoon Kim, Busan (KR); In-taek Hwang, Daejeon (KR); Kwang-yun Cho, Daejeon (KR); Jung-sup Choi, Daejeon (KR); Kwan-hwi Lee, Daejeon (KR); Bum-tae Kim, Daejeon (KR)

Correspondence Address: INTELLECTUAL PROPERTY LAW GROUP LLP, 12 SOUTH FIRST STREET, SUITE 1205, SAN JOSE, CA, 95113, US

(73) Assignee: GENOMINE, INC., POHANG, KYUNGBUK (KR)

(21) Appl. No.: 10/590,886

(22) PCT Filed: Feb. 18, 2005
(86) PCT No.: PCT/KR05/00454

§ 371 Date: Aug. 25, 2006

(51) Int. Cl. A01H 1/00; A01H 5/00; A01N 43/54; C07H 21/04; C07K 1/00; C12Q 1/68

(52) U.S. Cl. 800/294; 435/6; 504/239; 530/350; 536/23.6; 800/278

(57) ABSTRACT

The present invention discloses a polypeptide having function of cinnamyl alcohol dehydrogenase, a polynucleotide coding the polypeptide and those uses. Particularly, this present invention discloses a polypeptide having function of cinnamyl alcohol dehydrogenase, a polynucleotide coding the polypeptide, a method for inducing plant growth inhibition, a method for screening a compound inducing plant growth inhibition, and composition for inducing plant growth inhibition which comprises the compound obtained by the screening method.

### FIELD OF THE INVENTION

The present invention relates to a polypeptide having the cinnamyl alcohol dehydrogenase function, a polynucleotide encoding the polypeptide and those uses.

### BACKGROUND OF THE INVENTION

Lignin is biologically indispensable for plants and is material that is primarily responsible for the rigidity of plants.

Lignin also plays a role in enhancing disease resistance of plants by physically impeding the penetration and propagation of pathogenic agents.

All kinds of plants necessarily have the lignin biosynthesis pathway that doesn't exist in humans or animals, because of the roles of lignin as mentioned above.

Lignin biosynthesis is performed undergoing two successive pathways.

Firstly, L-phenylalanine, precursor, is converted to coumaric acid, ferulic acid or sinapic acid by the activities of enzymes such as phenylalanine ammonia-lyase, cinnamate 4-hydroxylase, 4-hydroxycinnamate 3-hydroylase, O-methyltransferase and ferulate 5-hydroxylase. This pathway is generally said to be the phenylpropanoid pathway, which is not a specific pathway for lignin biosynthesis.

Secondly, the produced acids are reduced to produce cinnamaldehydes, that is, coniferaldehyde, coumaldehyde and sinapaldehyde, by cinnamoyl CoA reductase, and then the cinnamaldehydes are converted to cinnamyl alcohols, that is, coniferyl alcohol, p-coumaryl alcohol and sinapyl alcohol, collectively referred to as momolignols, by cinnamyl alcohol dehydrogenase. This pathway is a specific pathway for lignin biosynthesis unlike the phenylpropanoid pathway. Lignin is biosynthesized from the cinnamyl alcohols by peroxidase, laccase and the like.

Therefore, the inhibition of the function of cinnamyl alcohol dehydrogenase, responsible for a specific pathway for lignin biosynthesis, may enable lignin not to be biosynthesized. This suggests that the inhibition of lignin biosynthesis may result in the inhibition of plant growth because lignin is biologically indispensable for plants.

The present inventors expected that the inhibition of lignin biosynthesis might result in the inhibition of plant growth in consideration of the fact that cinnamyl alcohol dehydrogenase is responsible for a specific pathway for lignin biosynthesis, and isolated the polypeptide and polynucletide having the function of the enzyme from Arabidopsis thaliana, and became to know that the prevention of the expression of the polypeptide by antisense method led to the growth of Arabidopsis thaliana.

This present has been provided, based on the above experimental result.

### BRIEF DESCRIPTION OF THE DRAWINGS

FIG. 1 depicts the results of SDS-PAGE analysis on each of fractions obtained from the cell extracts of E. coli transformant transformed with the recombinant vector containing the polynucleotide encoding a polypeptide having the cinnamyl alcohol dehydrogenase function, preferably the nucleotide sequence of SEQ ID. NO. 1.

FIG. 2a depicts the Lineweaver-Burk's blot showing the reaction velocity measured according to variation of substrate concentration, in order to measure the substrate-specific activity of the purified protein to coniferaldehyde, that is, a substrate of forward reaction.

FIG. 2b depicts the Lineweaver-Burk's blot showing the reaction velocity measured according to variation of substrate concentration, in order to measure the substrate-specific activity of the purified protein to coniferyl alcohol, that is, a substrate of backward reaction.

FIG. 3a depicts the schematic construction of a cloning vector before the polynucleotide encoding a polypeptide having the cinnamyl alcohol dehydrogenase function, preferably the nucleotide sequence of SEQ ID NO. 1 is inserted into the vector in an anti-sense direction.

FIG. 3b depicts the schematic construction of the recombinant vector into which the polynucleotide encoding a polypeptide having the cinnamyl alcohol dehydrogenase function, preferably the polynucleotide of SEQ ID. NO. 1 is inserted in an anti-sense direction.

FIG. 4a depicts Atcad-H1, the plant sapling germinated from a seed of Arabidopsis thaliana transformant into which the anti-sense nucleotide against a polynucleotide encoding a polypeptide having the cinnamyl alcohol dehydrogenase function, preferably the polynucleotide of SEQ ID. NO. 1 is introduced.

FIG. 4b depicts Atcad-H6, the plant sapling from a seed of Arabidopsis thaliana transformant into which the anti-sense nucleotide against a polynucleotide encoding a polypeptide having the cinnamyl alcohol dehydrogenase function, preferably the polynucleotide of SEQ ID. NO. 1 is introduced.

### DETAILED DESCRIPTION OF THE INVENTION

Technical Theme

The object of the present invention is to provide a polypeptide having the cinnamyl alcohol dehydrogenase function.

Another object of the present invention is to provide a polynucleotide encoding the polypeptide.

Still another object of the present invention is to provide a method for inhibiting plant growth.

Still another object of the present invention is to provide a process for screening a growth inhibitor of plants.

Still another object of the present invention is to provide a composition for inhibiting plant growth comprising the growth inhibitor screened by the process.

Other objects or aspects of the present invention are set forth hereinafter.

Technical Solution

In one aspect, the present invention provides a polypeptide having the cinnamyl alcohol dehydrogenase function.

The function of the polypeptide was confirmed through the following process.

Firstly, as demonstrated in the following Examples, full-length cDNA of Arabidopsis thaliana was constructed by using the primers manufactured based upon the nucleotide sequence of the gene that is deduced to encode the polypeptide having the cinnamyl alcohol dehydrogenase function in Arabidopsis thaliana (GeneBank accession number NM 121949). Secondly, the molecular weight of the polypeptide encoded by the cDNA was estimated by analyzing the open reading frame on the basis of the nucleotide sequence the cDNA, and it was identified that the polypeptide having the estimated molecular weight was expressed in E. coli. transformed with the recombinant vector containing the cDNA fragment. Further, it was identified that the polypeptide expressed in E. coli participates in converting coniferaldehyde to coniferyl alcohol.

Therefore, as used herein, it is preferable that the cinnamyl alcohol dehydrogenase of the present invention defines the enzyme participating in biosynthesis of coniferyl alcohol of three kinds of monolignols, that is to say, coniferyl alcohol, p-coumaryl alcohol and sinapyl alcohol, and having substrate specificity for coniferaldehyde. More preferably, the cinnamyl alcohol dehydrogenase of the present invention defines the enzyme having substrate specificity for coniferyl alcohol, a substrate of backward reaction, as well as coniferaldehyde, a substrate of forward reaction. Most preferably, the cinnamyl alcohol dehydrogenase of the present invention defines the enzyme having higher substrate affinity for coniferaldehyde than coniferyl alcohol.

In detail, the polypeptide having the cinnamyl alcohol dehydrogenase function according to the present invention is a member selected from a group consisting of (a), (b) and (c) polypeptide:
(a) polypeptide containing all portion of the amino acid sequence set forth in SEQ ID NO. 2;
(b) polypeptide containing a substantial portion of the amino acid sequence set forth in SEQ ID NO. 2;
(c) polypeptide substantially similar to the above (a) or (b) polypeptide.

As used herein, a “polypeptide containing a substantial portion of the amino acid sequence set forth in SEQ ID. NO. 2” refers to a polypeptide containing the partial portion of amino acid sequence of SEQ ID NO. 2 that still retains the cinnamyl alcohol dehydrogenase function, compared with a polypeptide consisting of the amino acid sequence of SEQ ID NO. 2. A polypeptide retaining the cinnamyl alcohol dehydrogenase function is regarded as the polypeptide of the present invention, regardless of the polypeptide's length or activity degree. That is to say, any polypeptide retaining the cinnamyl alcohol dehydrogenase function can be a “polypeptide containing a substantial portion of the amino acid sequence set forth in SEQ ID NO. 2” even if it is shorter in the length or lower in the enzymatic activity than the polypeptide consisting of the amino acid sequence of SEQ ID NO. 2. Even though any amino acid sequence is partially deleted from or added to the amino acid sequence of SEQ ID NO. 2, those skilled in the arts may expect that the deleted or added polypeptide can still retains the cinnamyl alcohol dehydrogenase function. For example, the polypeptides that are deleted in the N-terminus and/or the C-terminus belong to such a polypeptide. It is already well known in prior arts that polypeptides can often retain the intrinsic activity, even though deleted in the N-terminus and/or the C-terminus. Depending upon cases, the polypeptide deleted in the N-terminus and/or the C-terminus may not retain the enzymatic activity, since the termini are an essential motif of enzyme, but those skilled in the art can discriminate or detect the active polypeptide from inactive polypeptides within the ordinary knowledge. Also, even if any other region as well as the termini is deleted, the deleted polypeptides can still retain the intrinsic activity. Those skilled in this art can decide whether the deleted polypeptides still retain the intrinsic activity or not, within the ordinary knowledge. Particularly, the nucleotide sequence of SEQ ID NO. 1 and the amino acid sequence of SEQ ID NO. 2 are disclosed in the specification. The polypeptide that is encoded by the nucleotide sequence of SEQ ID NO. 1 and consists of the amino acid sequence of SEQ ID NO. 2 is clarified to have the cinnamyl alcohol dehydrogenase function in the following Examples. Therefore, it is obvious that those skilled in the art can identify easily whether or not the polypeptide partially deleted in the amino acid sequence of SEQ ID NO. 2 still retain the intrinsic activity of the polypeptide consisting of the amino acid sequence of SEQ ID NO. 2, within the ordinary knowledge. Hence, it is naturally understood that a “polypeptide containing a substantial portion of the amino acid sequence set forth in SEQ ID NO. 2” includes all deleted polypeptides that still retain the cinnamyl alcohol dehydrogenase function and can be easily manufactured by those skilled in the art within the ordinary knowledge on a basis disclosed in the specification.

As used herein, a “polypeptide substantially similar to above (a) or (b) polypeptide” refers to a polypeptide that still retains the function of polypeptide consisting of the amino acid sequence of SEQ ID NO. 2, that is, the cinnamyl alcohol dehydrogenase function, even if one or more amino acids are substituted. At this time, insofar as any polypeptide still retains the cinnamyl alcohol dehydrogenase function, the polypeptide's activity or substitution degree is of little importance. That is to say, any substituted polypeptide still retaining the cinnamyl alcohol dehydrogenase function can be within the scope of the present invention, even though the polypeptide has many substituted amino acids or is much lower activity than that of the polypeptide consisting of the amino acid sequence of SEQ ID NO. 2. In reference, even if one more amino acids are substituted, the polypeptide substituted in one more amino acids may retain the intrinsic activity of a polypeptide if the substituting amino acids are chemically equivalent to the substituted amino acids. If a hydrophobic amino acid such as alanine is displaced with other hydrophobic amino acids such as glycin or more hydrophobic amino acids such as valine, leucine or isoleucine, the polypeptide substituted in one more amino acids may retain the intrinsic function of a polypeptide, in spite of activity reduction. In addition, if a negative-charged amino acid such as glutamic acid is displaced with other negative-charged amino acids such as aspartic acid, the polypeptide substituted in one more amino acids may retain the intrinsic function of a polypeptide. Besides, if a positive-charged amino acid such as arginine is displaced with other positive-charged amino acids such as lysine, the polypeptide substituted in one more amino acids may retain the intrinsic function of a polypeptide, in spite of activity reduction. Furthermore, even if the N-terminus or the C-terminus is substituted, the polypeptide can retain the intrinsic activity. Those skilled in this art can easily manufacture the polypeptide that still retains the function of the polypeptide consisting of the amino acid sequence of SEQ ID NO. 2, even if substituted in one more amino acids, and decide whether or not this polypeptide still retains the intrinsic activity. Particularly, the nucleotide sequence of SEQ ID NO. 1 and the amino acid sequence of SEQ ID NO. 2 are disclosed in the specification, and the polypeptide consisting of the amino acid sequence of SEQ ID NO. 2 is confirmed to have the cinnamyl alcohol dehydrogenase function in the following Examples. Therefore, it is comprehended that those skilled in the art can accomplish a “polypeptide substantially similar to above (a) or (b) polypeptide” easily. Also in the present invention, a “polypeptide substantially similar to above (a) or (b) polypeptide” includes all polypeptides that is substituted in one more amino acids but retains the cinnamyl alcohol dehydrogenase function.

As described in the above, a “polypeptide substantially similar to above (a) or (b) polypeptide” means all polypeptides that are substituted in one more amino acids and still retains the cinnamyl alcohol dehydrogenase function. However, judging from the enzymatic activity, the polypeptide is preferable to become higher in the sequence homology, compared with the amino acid sequence of SEQ ID NO. 2. Preferably, the polypeptide has over 60% of sequence homology in the minimum while it has exactly 100% of sequence homology in the maximum.

In detail, the above sequence homology is preferable to become higher in the order of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 99.6%. At this moment, a polypeptide having 99.6% of sequence homology means a polypeptide substituted in one amino acid, compared with the amino acid sequence of SEQ ID NO. 2.

Because a “polypeptide substantially similar to above (a) or (b) polypeptide” includes not only a “polypeptide substantially similar to the polypeptide containing all portion of the amino acid sequence set forth in SEQ ID NO. 2” but also a “polypeptide substantially similar to the polypeptide containing a substantial portion of the amino acid sequence set forth in SEQ ID NO. 2,” all the above-described description applies to a “polypeptide substantially similar to the polypeptide containing all portion of the amino acid sequence set forth in SEQ ID NO. 2” but also a “polypeptide substantially similar to the polypeptide containing a substantial portion of the amino acid sequence set forth in SEQ ID NO. 2.”

In another aspect, the present invention provides an isolated polynucleotide encoding above-mentioned polypeptide. At this moment, an “above-mentioned polypeptide” has the meaning to include the polypeptide that has the cinnamyl alcohol dehydrogenase function and contains all portion of the amino acid sequence set forth in SEQ ID NO. 2, the polypeptide that has the cinnamyl alcohol dehydrogenase function and contains a substantial portion of the amino acid sequence set forth in SEQ ID NO. 2, and the polypeptide that has the the cinnamyl alcohol dehydrogenase function and is substantially similar to any one of above-mentioned polypeptide. The “above-mentioned polypeptide” also includes all preferable polypeptides described above. Therefore, the polynucleotide of the present invention includes an isolated polynucleotide encoding the polypeptide that has the cinnamyl alcohol dehydrogenase function and contains all portion of the amino acid sequence set forth in SEQ ID NO. 2, an isolated polynucleotide encoding the polypeptide that has the cinnamyl alcohol dehydrogenase f unction and contains a substantial portion of the amino acid sequence set forth in SEQ ID NO. 2, and an isolated polynucleotide encoding the polypeptide that has the cinnamyl alcohol dehydrogenase function and is substantially similar to any one of above-mentioned polypeptide. The polynucleotide of the present invention further includes the isolated polynucleotide encoding all the polypeptides that have the cinnamyl alcohol dehydrogenase function and have the sequence homology in the above-illustrated order. It is natural that those skilled in the art can manufacture conveniently the polynucleotide encoding the amino acid sequence on a basis of its sequence if available.

On the other hand, an “isolated polynucleotide” defines to include a polynucleotide synthesized chemically, a polynucleotide separated from organism such as Arabidopsis thaliana and a polynucleotide containing modified nucleotides, and also include single-stranded or double-stranded RNA or DNA polymers. Particularly, an “isolated polynucleotide” includes genomic DNA separated from Arabidopsis thaliana as well as cDNA and chemically synthesized polynucleotide. It is natural within the common knowledge that those skilled in the art can synthesize a polynucleotide chemically, manufacture cDNA, purify genomic DNA and the like, on a basis of prior arts, the amino acid sequence of SEQ ID NO. 2, and the nucleotide sequence of SEQ ID NO. 1 encoding the same as disclosed in the specification.

In another aspect, the present invention provides an anti-sense nucleotide that can bind complementarily to above-mentioned polynucleotide.

The anti-sense nucleotide of the present invention includes all poly (or oligo) nucleotides that can bind complementarily against a polynucleotide so as to inhibit the transcription (in DNA) or the translation (in RNA). If the anti-sense nucleotide binds complementarily against a polynucleotide encoding a polypeptide having the cinnamyl alcohol dehydrogenase function to inhibit the transcription (in DNA) or the translation (in RNA), its length and complementary sequence homology is of little matter. Even a small polynucleotide comprising less than 30 nucleotides may be used as an anti-sense nucleotide, if it has 100% complementary sequence homology against its target sequence and is used under a proper condition by adjusting concentration, pH or the like. Even a complementary sequence under 100% complementary sequence homology may also become an anti-sense nucleotide, if it has a proper length. Therefore, it is natural that the anti-sense nucleotides of the present invention includes all anti-sense nucleotides that can retain the anti-sense activity to inhibit the transcription or the translation, regardless of length and complementary sequence homology degree.

It is natural within the common knowledge that those skilled in the art can determine the proper length and complementary sequence homology of an anti-sense nucleotide, and manufacture the anti-sense nucleotide on a basis of prior arts, the nucleotide sequence of SEQ ID NO. 1 and the amino acid sequence of SEQ ID NO. 2 as disclosed in the specification.

Preferably, the anti-sense nucleotide of the present invention can be an anti-sense nucleotide containing the partially complementary region against the nucleotide sequence of SEQ ID NO. 1. It is understood in the present invention that an “anti-sense nucleotide containing partially complementary region against the nucleotide sequence of SEQ ID NO. 1” means an anti-sense nucleotide that is sufficiently long to bind complementarily against DNA consisting of the nucleotide sequence of SEQ ID NO. 1 or RNA transcribed from the same so as to inhibit the transcription or the translation, as illustrated above.

In another aspect, the present invention provides a recombinant vector containing above-mentioned polynucleotide and a transformant transformed with the recombinant vector.

As described in following Examples, the polynucleotide that consists of the nucleotide sequence of SEQ ID NO. 1 and encodes the polypeptide having the cinnamyl alcohol dehydrogenase function is introduced to the cloning vector pCAL-n (Stratagene, USA) to construct the recombinant vector pCAtCAD-H.

After that, the recombinant vector pCAtCAD-H is transformed to E. coli and the resulting transformant is cultivated and induced to express a polypeptide in order to determine the molecular weight of the polypeptide. As a result, the polypeptide is identified to be the same in the molecular weight with the polypeptide estimated from the open reading frame of SEQ ID NO. 1.

In a preferred embodiment, the present invention provides the recombinant vector pCAtCAD-H and the E. coli transformant that is transformed with the recombinant vector.

In another aspect, the present invention provides a method for inhibiting plant growth. In detail, the method for inhibiting plant growth comprises step of inhibiting the expression or function of a polypeptide that has the cinnamyl alcohol dehydrogenase function and consists of the amino acid sequence of SEQ ID NO. 2 or its equivalent sequence.

As used herein, “inhibiting plant growth” defines “causing retard of plant growth or plant lethality”

As demonstrated above, the cinnamyl alcohol dehydrogenase is an enzyme participating in a specific pathway for lignin biosynthesis, and lignin is indispensable for plants. Therefore, if the cinnamyl alcohol dehydrogenase is not expressed and functionally suppressed, the plant growth may be inhibited because lignin biosynthesis is inhibited. As disclosed in following Examples, Arabidopsis thaliana is transformed by using an anti-sense nucleotide complementary to the nucleotide sequence of SEQ ID NO. 1 and observed to retard in the growth or the like. Therefore, the method for inhibiting plant growth of the present invention adopts the step of inhibiting the expression of a polypeptide having the cinnamyl alcohol dehydrogenase function or suppressing the function thereof.

As used herein, a “polypeptide consisting of the amino acid sequence of SEQ ID NO. 2 or its equivalent sequence” means to include all polypeptides that are derivatives to the polypeptide consisting of the amino acid sequence of SEQ ID NO. 2, and retain the cinnamyl alcohol dehydrogenase function and evolve according to plant sorts to contain the amino acid sequence varied from the amino acid sequence of SEQ ID NO. 2. Therefore, it is naturally understood in the present invention that the scope of plant in the method for inhibiting plant growth may include not only Arabidopsis thaliana but also any other plants, even though the polypeptide consisting of the amino acid sequence of SEQ ID NO. 2 is isolated form Arabidopsis thaliana. The polypeptide consisting of the amino acid sequence of SEQ ID NO. 2 or its equivalent sequence is preferable to become higher in the sequence homology, naturally most preferable to have 100% of sequence homology, compared with the amino acid sequence of SEQ ID NO. 2. However, the polypeptide is preferable to have at least 60% of sequence homology in the minimum.

In detail, the above sequence homology is preferable to become higher in the order of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In the meantime, it is natural that those skilled in the art can exploit prior arts to inhibit the expression of polypeptide, preferably by using insertion of anti-sense nucleotide, gene deletion, gene insertion, T-DNA insertion, homologous recombination or transposon tagging, small interfering RNA (siRNA) and the like.

As described in the following Examples, in order to inhibit the expression of polypeptide, the anti-sense nucleotide is introduced to a plant by the process comprising steps: (1) manufacturing an anti-sense nucleotide against the polynucleotide of SEQ ID NO. 1; (2) constructing the recombinant vector containing the same; (3) transforming the recombinant vector to Agrobacterium tumefaciens; and (4) transforming the resulting transformant to Arabidopsis thaliana. As the result of cultivating a seed of Arabidopsis thaliana transformant, it is confirmed that the transformant retards in the growth remarkably, causes seriously yellows in whole leaves, becomes higher in the lethality or the like (See Example 3).

Therefore, in the method for inhibiting plant growth, preferably an anti-sense nucleotide containing a partially complementary region against the nucleotide sequence of SEQ ID NO. 1 can be introduced to a target plant in order to inhibit the expression of polypeptide. More preferably, a transformant transformed with the recombinant vector containing an anti-sense nucleotide against the nucleotide sequence of SEQ ID NO. 1 can be introduced into a target plant. Most preferably, the transformant can be Agrobacterium tumefaciens transformed with the recombinant vector containing the same. At this moment, an “anti-sense nucleotide containing partially complementary region against the nucleotide sequence of SEQ ID NO. 1” is the same as already described above in connection with the anti-sense nucleotide of the present invention.

Generally, it is reported that an anti-sense nucleotide may bind onto a target nucleotide array within a nucleotide sequence (RNA or DNA) to inhibit the function of nucleic acids or the expression. That is to say, the anti-sense nucleotide against specific gene sequence can hybridize both RNA and DNA so as to inhibit the expression of specific gene in transcription or translation.

Therefore, if the polypeptide having the cinnamyl alcohol dehydrogenase function does not work properly by inhibiting the expression or function of the polypeptide consisting of the amino acid sequence of SEQ ID NO. 2 or its equivalent sequence, that can result in inhibiting the plant growth because lignin biosynthesis is inhibited.

The method for inhibiting plant growth of the present invention is harmless to human or animals, since it adopts a mechanism to hinder the biosynthetic pathway of lignin that exists in plants but is missed in human or animals, to inhibit the production of lignin.

In another aspect, the present invention provides a process for screening a growth inhibitor of plants, which comprises the step of screening a substance inhibiting the expression or function of a polypeptide having the cinnamyl alcohol dehydrogenase function and consisting of the amino acid sequence of SEQ ID NO. 2 or its equivalent sequence.

As used herein, a “polypeptide having the cinnamyl alcohol dehydrogenase function and consisting of the amino acid sequence of SEQ ID NO. 2 or its equivalent sequence” is the same as already explained in connection with the method for inhibiting plant growth according to the present invention.

Preferably, the growth inhibitor of the present invention can be an anti-sense nucleotide containing a partially complementary region against the nucleotide sequence of SEQ ID NO. 1; more preferably, a transformant transformed with the recombinant expression vector comprising the anti-sense nucleotide containing a partially complementary region against the nucleotide sequence of SEQ ID NO. 1; and most preferably, Agrobacterium tumefaciens transformant transformed with the recombinant expression vector. At this moment, an “anti-sense nucleotide containing partially complementary region against the nucleotide sequence of SEQ ID NO. 1” is the same as already described above in connection with the anti-sense nucleotide of the present invention.

In another aspect, the present invention provides a composition for inhibiting plant growth comprising the growth inhibitor screened by the above-described screening process.

This composition will have an effect on inhibiting plant growth, without harm to animal or human. This suggests that the composition may become an environment-preservative herbicide harmless to human.

Meanwhile, the growth inhibitor can be selected from a group comprising (1) an anti-sense nucleotide as described above, particularly anti-sense nucleotide containing partially complementary region against the nucleotide sequence of SEQ ID NO. 1; (2) a recombinant vector containing the anti-sense nucleotide; and (3) a transformant transformed with the recombinant vector, and preferably, Agrobacterium tumefaciens transformed with the recombinant vector. At this moment, the “anti-sense nucleotide containing partially complementary region against the nucleotide sequence of SEQ ID NO. 1” is the same as already described above in connection with the anti-sense nucleotide of the present invention.

Peculiar Effect

According to the present invention, the polypeptide having the cinnamyl alcohol dehydrogenase function, the polynucleotide encoding the polypeptide, the method for inhibiting plant growth, the process for screening a growth inhibitor of plants, and the composition for inhibiting plant growth comprising the growth inhibitor screened by the process are provided.

### BEST MODE FOR CARRYING OUT THE INVENTION

Hereinafter, the present invention will be described in reference to preferred embodiments. However, the scope of the present invention is not limited to these embodiments.

### Example 1

### Separation of Polynucleotide Encoding Polypeptide having the Cinnamyl Alcohol Dehydrogenase Function from Arabidopsis thaliana

In order to separate a gene encoding a polypeptide having the cinnamyl alcohol dehydrogenase function, the screening is performed by using Arabidopsis thaliana as explained below.

<1-1> Cultivation and Culture of Arabidopsis thaliana

Arabidopsis thaliana was cultivated in a soil-filled pot or cultivated in a petri dish by using MS medium (Murashige and Skoog salts, Sigma, U.S.A.) containing 2% sucrose (pH 5.7) and 0.8% agar. In this study, vitamin B6 group (including pyridoxine) was excluded from the vitamin mixture used in the MS medium. The culture pot was incubated at 22° C. in a growth chamber and controlled by 16/8 hours of light and dark cycle.

<1-2> RNA Purification and Construction of cDNA Library

In order to construct cDNA library, Arabidopsis thaliana leaves were collected in various stages of differentiation to separate total RNAs by using TRI reagent (Sigma, USA). Then, total RNAs were purified to obtain poly(A)+ RNA according to the protocol of mRNA purification kit (Pharmacia, USA). The poly(A)+ RNA was utilized to prepare the double stranded cDNA by Not I-(dT)₁₈ as primer through cDNA synthesis kit (Time Saver, Pharmacia, USA).

<1-3> Purification of Gene Encoding the Polypeptide having the Cinnamyl Alcohol Dehydrogenase Function

In order to separate gene encoding the polypeptide having the cinnamyl alcohol dehydrogenase function, the primer of SEQ ID NO. 3 containing the recognition site of restriction enzyme BamHI and the reverse primer of SEQ ID NO. 4 containing the recognition site of restriction enzyme HindIII were synthesized, based upon the nucleotide sequence of the gene that is deduced to encode the polypeptide having the cinnamyl alcohol dehydrogenase function in Arabidopsis thaliana (GeneBank accession number NM 121949). By performing PCR using the resulting primers, the full-length cDNA was amplified and isolated from the cDNA library of Arabidopsis thaliana constructed in Example 1-2.

As the result of analyzing the isolated cDNA, it is identified that the cDNA sequence contains the open reading frame (ORF) in 981 bps size, encodes 326 amino acids totally having 35.6 kDa molecular weight and is composed of 6 exons and 5 introns. The gene of the present invention has been named as AtCAD-H (Arabidopsis thaliana cinnamyl alcohol dehydrogenase H) (Hereinafter, the protein and gene is referred to as AtCAD-H or AtCAD-H gene and AtCAD-H or AtCAD-H protein respectively). The isoelectric point of the AtCAD-H protein encoded by the gene is observed to be 7.15.

### Example 2

### Purification of Protein Expressed by AtCAD-H Gene in E. coli

<2-1> Induction of Protein Expression

The DNA fragment containing full-length cDNA region of AtCAD-H gene that was amplified and isolated in Example 1-3 was digested by using the restriction enzymes BamHI and HindIII and inserted to the recognition site of restriction enzymes BamHI and HindIII in the cloning vector pCAL-n (Stratagene, USA) to construct the recombinant vector pCAtCAD-H. At this moment, the cloning vector pCAL-n is advantageous to contain the tag sequence of calmodulin-binding peptide and the exogenous protein expressed can be separated easily by using a calmodulin resin.

After that, the recombinant vector pCAtCAD-H was transformed into E. coli to amplify, retransformed to E. coli BL21-Gold (DE3) (Stratagene, USA), and cultivated at 37° C. with agitation at 150 rpm in LB broth (Luria-Bertani broth, USB, USA) containing 100 μg/ml of ampicillin until O.D. 600 value reached 0.7. In order to induce the intracellular expression of target protein in E. coli, isopropyl-D-thiogalactoside (IPTG) was added to the cell suspension to adjust final concentration to 1 mM and cultivated for 2 hours. The resulting cell was washed by using 50 mM potassium phosphate buffer (pH 7.0) containing 50 mM of MgSO₄ and 0.4 M of NaCl, and centrifuged at 4,000 ×g for 15 minutes, and then the precipitate was collected to be stored at −20° C.

<2-2> Protein Purification

The cell precipitate isolated in Example 2-1 was suspended in CaCl₂ binding buffer (50 mM Tris-HCl, pH 8.0, 150 mM NaCl, 10 mM β-mercaptoethanol, 1.0 mM magnesium acetate, 1.0 mM imidazole, 2 mM CaCl₂). Lysozyme was added to the cell suspension for the final concentration to be adjusted to 200 μg/ml, and then the resulting suspension was centrifuged for 15 minutes, and ultrasonicated for 30 seconds. The resulting samples were cooled down with ice for 5 minutes and this procedure (cooling after ultrasonication) was further performed two times. The samples were centrifuged at 10,000×g for 5 minutes to obtain supernatants. The supernatants were purified using calmodulin affinity chromatography. That is to say, the supernatants (crude extracts) were applied onto the equilibrated calmodulin affinity chromatography resin, followed by the reaction at 4° C. for 24 hours. In order to remove unattached proteins and other substances, the column was washed with CaCl₂ binding buffer. The proteins attached to the calmodulin were isolated from column matrix using elution buffer (50 mM Tris-HCl, pH 8.0, 10 mM β-mercaptoethanol, 2 mM EDTA, 150 mM NaCl). The proteins isolated from crude extracts of E. coli transformed with pCAL-n vector were used as a control group.

In order to identify the purification of the protein, SDS-PAGE analysis was performed for the fractions 1 to 13 of all effluents separated from E. coli transformed with pCAtCAD-H recombinant vector.

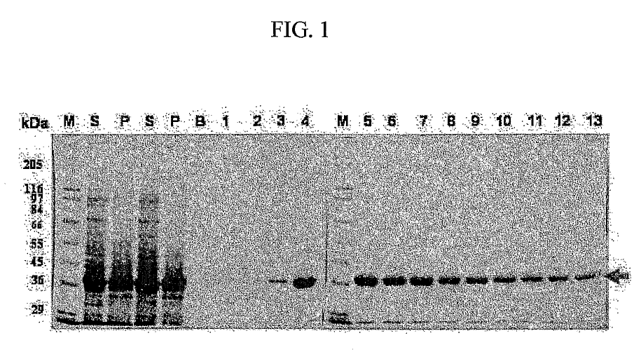

As a result, as shown in FIG. 1, it was confirmed that the fractions 4 to 9 of all the fractions separated from E. coli transformed with pCAtCAD-H recombinant vector had the highest protein content and that the effluents separated from E. coli transformed with pCAtCAD-H recombinant vector contained fusion proteins of 39.6 kDa size (35.6 kDa (molecular weight of the protein expressed by AtCAD-H gene)+4 kDa (molecular weight of calmodulin binding peptide)). On the other hand, it was confirmed that the effluents separated from the control group didn't contain such size protein (Data is not presented).

In FIG. 1, the fusion protein in 39.6 kDa size is indicated by arrow (←) and M is an abbreviation of a “marker.” Lanes S is an supernatants obtained from E. coli transformant transformed with the recombinant vector containing AtCAD-H gene; lane P, insoluble protein obtained from E. coli transformant transformed with the recombinant vector containing AtCAD-H gene; lane B, fraction directly before the calmodulin affinity chromatography was eluted using an elution buffer; and lane 1 to 13, fractions 1 to 13 of an extract of E. coli transformed with the recombinant vector containing AtCAD-H gene.

### Example 3

### Analysis on the Enzyme Activity of the Protein

In order to identify whether the isolated protein has the cinnamyl alcohol dehydrogenase function related to lignin biosynthesis, the enzyme activity in forward reaction was measured using coniferaldehyde as a substrates. Also, the enzyme activity in backward reaction was measured using coniferyl alcohol as a substrates so as to identify whether the enzyme also involves in backward reaction or not.

The enzyme activities were measured at 405 nm using a microplate reader (Bio-rad Backman Microplate Reader) adjusted at 37° C. according to the method of Mitchell et al. (Mitchell et al. Planta, 208: 31-47, 1998). In 200 ml of the reaction solution for the measurement of the enzyme activity in forward reaction, 100 mM Tris-buffer (Tris-HCl pH 9.3), 100 mM NADPH, and 2-3 μg of the protein purified in Example 2-2 were contained. The conditions of the reaction solution for the measurement of the enzyme activity in backward reaction is the same as that of the reaction solution of forward reaction, except that 100 mM NADPH⁺ was contained in place of 100 mM NADPH.

Coniferaldehyde and coniferyl alcohol at the various concentrations were added to the forward and backward reaction solutions respectively to measure the enzyme activity. The well containing only other substances except the purified protein was used as a control group.

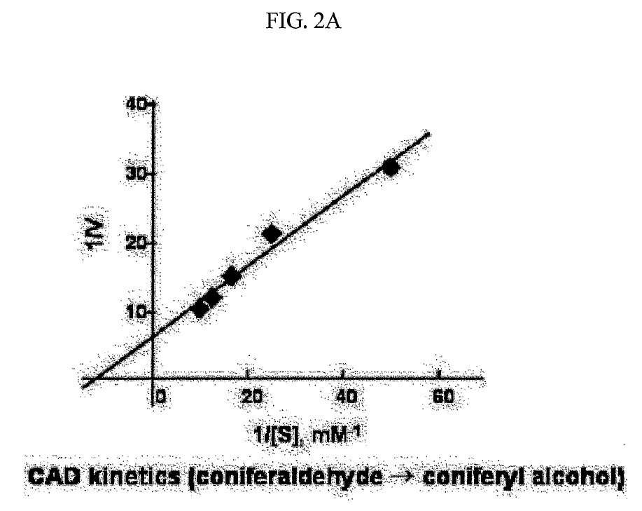

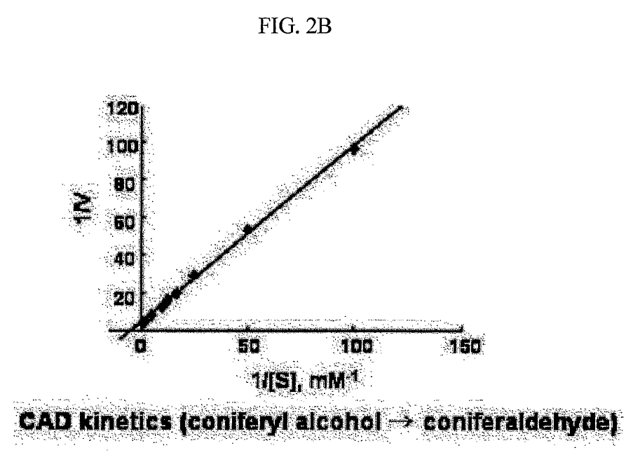

The enzyme activity of the purified protein was suitable for Michaelis-Menten kinetics for 100 mM coniferaldehyde, that is, the substrate of forward reaction. The reaction velocities were measured according to variation of substrate concentration, and represented in a Lineweaver-Burk's plot (see FIG. 2a). As a result, Kₘ and Vₘₐₓ values were identified to be 1.98×10⁻⁵M and 0.238 respectively. These results indicated that the protein expressed from AtCAD-H gene is cinnamyl alcohol dehydrogenase having substrate specificity to coniferaldehyde among cinnamaldehyde as a substrate.

Meanwhile, the enzyme activity of the purified protein was suitable for Michaelis-Menten kinetics for 100 mM coniferyl alcohol, that is, the substrate of backward reaction. The reaction velocities were measured according to variation of substrate concentration, and represented in a Lineweaver-Burk's plot (see FIG. 2b). As a result, Kₘ and Vₘₐₓ values were identified to be 3.74×10⁻⁴M and 0.102 respectively. These results indicated that the substrate affinity in forward reaction is higher than that in backward reaction. This suggests that AtCAD-H protein may have a self-regulation function to maintain the proper concentration of cinnamyl alcohol in lignin biosynthesis. Consequently, it is expected that the protein of the present invention plays an important role in lignin biosynthesis.

Meanwhile, the study has revealed that the most suitable pH ranges of the present protein in forward and backward reactions are not different each other. This suggests that the pH change in a living organism doesn't not almost affect the activities of the present protein in forward and backward reactions

### Example 4

### Preparation and Characterization of Arabidopsis thaliana Transformant Inserted with Anti-Sense Construct against AtCAD-H Gene

<4-1> Preparation of Arabidopsis Transformant Inserted with Anti-Sense Construct against AtCAD-H Gene

In order to examine the physiological characteristics of protein isolated in the above Example 2-2, Arabidopsis thaliana transformant into which the AtCAD-H gene is inserted in the anti-sense direction was prepared to suppress the expression of AtCAD-H transcript.

AtCAD-H cDNA was amplified in the anti-sense direction through PCR from Arabidopsis thaliana cDNA, by using a forward primer of SEQ ID NO. 5 containing the recognition site of restriction enzyme BglII and reverse primer of SEQ ID NO. 6 containing the recognition site of restriction enzyme XbaI. The resulting DNA fragment was digested by restriction enzyme BglII and XbaI, ligated to the cloning vector pSEN controlled by the promoter of sen 1 that is a stress- or aging-related gene to prevent plant death during the germination, and the recombinant vector pSEN-antiAtCAD-H as an anti-sense construct against AtCAD gene was manufactured. sen 1 promoter is specific for a plant gene expressed according to growth stages.

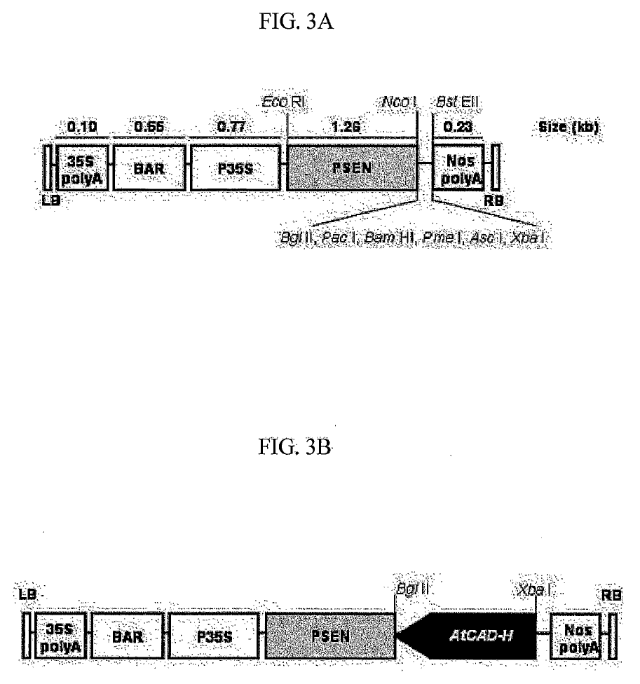

Meanwhile, in FIG. 3, the construction of the cloning vector pSEN (See FIG. 3a) and the recombinant vector pSEN-antiAtCAD-H (See FIG. 3b) were illustrated. As depicted in FIG. 3, BAR indicates the bar gene conferring resistance to herbicide BARSTA (phosphinothricin acetyltransferase gene); RB, right border; LB, left border; P35S, CaMV 35S RNA promoter; 35S poly A, CaMV 35S RNA poly A; PSEN, sen 1 promoter; Nos poly A, poly A of nopaline synthase gene.

The recombinant vector pSEN-AtPDZ4 was introduced into Agrobacterium tumefaciens by the electroporation method. Agrobacterium transformant was cultivated at 28° C. until OD value reached 1.0 at 600 nm, centrifuged at 25° C. for 10 minutes at 5,000 rpm, and then cells were harvested. The resulting cells were suspended by using infiltration medium (IM; 1×MS salts, 1×B5 vitamin, 5% sucrose, 0.005% Silwet L-77, Lehle Seed, USA) until final OD value reached 2.0 at 600 nm. 4-weeked Arabidopsis thaliana was submerged to Agrobacterium tumefaciens suspension in a vacuum chamber, left for 10 minutes under 10⁴ Pa of vacuum, and then put in a polyethylene bag for 24 hours. After that, Arabidopsis tumefaciens transformant was cultivated continuously to harvest seed T1. At this moment, wild type Arabidopsis thaliana and Arabidopsis thaliana transformant transformed with the cloning vector pSEN without AtCAD-H gene were adopted as control groups.

<4-2> Characterization of Arabidopsis thaliana Transformant T1 and T2

The resulting seed harvested from Arabidopsis thaliana transformant as described in Example 4-1 was submerged in herbicide solution, Basta (Kyungnong Co. Ltd., Korea) for 30 minutes, cultivated and selected.

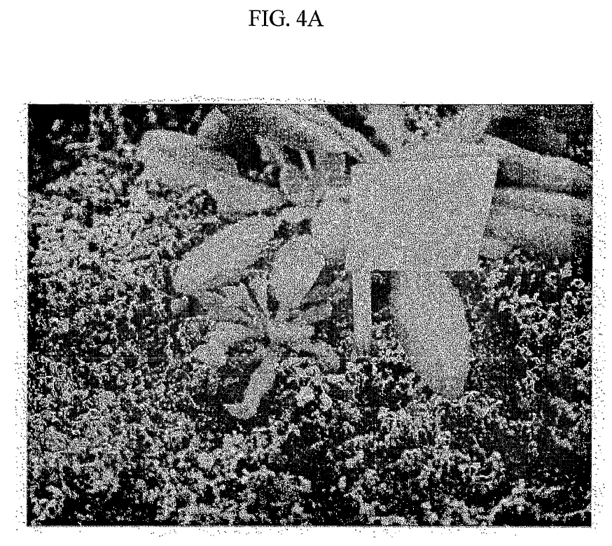

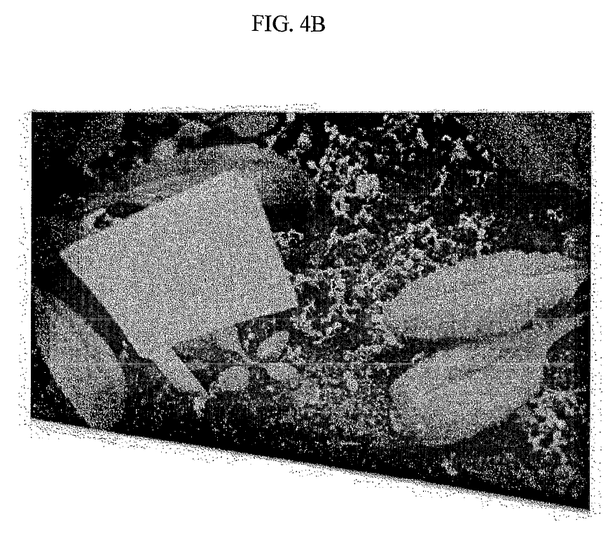

As a result, compared with the control group (Arabidopsis transformed with the cloning vector pSEN without anti-sense AtCAD-H gene), Atcad-H1 of Arabidopsis thaliana transformants was suppressed severely to grow and gave rise to a deep green phenomenon in leaves (See FIG. 4a), and Atcad-H6 of Arabidopsis thaliana transformants was suppressed severely to grow like the above Atcad-H1 and gave rise to a change (wilt) in a leaf shape (See FIG. 4b).

It is presumed that this trivial phenotypic difference between two lines is caused by the difference of antisense effect against the present gene. Afterward, Arabidopsis thaliana transformant was observed to have undergone fatal damage in flowering and seed production. Also, it was observed that the phenotypic change like the above was generated in the mutants in which the present gene was silenced by T-DNA tagging method.

What is claimed is:

1. A polypeptide. having the cinnamyl alcohol dehydrogenase function, selected from a group comprising (a), (b) and (c) polypeptide:
(a) polypeptide containing all portion of the amino acid sequence set forth in SEQ ID NO. 2;
(b) polypeptide containing a substantial portion of the amino acid sequence set forth in SEQ ID NO. 2;
(c) polypeptide substantially similar to the above (a) or (b) polypeptide,
wherein the cinnamyl alcohol dehydrogenase has substrate specificity for coniferyl alcohol, a substrate of backward reaction as well as coniferaldehyde, a substrate of forward reaction, and has higher substrate affinity for coniferaldehyde than coniferyl alcohol.

2. A polynucleotide encoding the polypeptide of claim 1.

3. A method for inhibiting a plant growth, which comprises a step of inhibiting the expression or function of a polypeptide that has the cinnamyl alcohol dehydrogenase function and consists of the amino acid sequence of SEQ ID NO. 2 or its equivalent sequence.

4. The method according to claim 3, in which the step is performed by introducing an anti-sense nucleotide against the polynucleotide set forth in claim 2 into a plant.

5. The method according to claim 3, in which the step is performed by introducing a recombinant vector containing an anti-sense nucleotide against the polynucleotide set forth in claim 2 into a plant.

6. The method according to claim 3, in which the step is performed by introducing a Agrobacterium tumefaciens transformant transformed with a recombinant vector containing an anti-sense nucleotide against the polynucleotide set forth in claim 2, into a plant.

7. The method according to claim 3, in which the step is performed by any one technique that is selected among gene deletion, gene insertion, T-DNA insertion, homologous recombination, transposon tagging, small interfering RNA (siRNA) and the like.

8. A process for screening a growth inhibitor of plants, which comprises a step for screening a substance inhibiting the expression or function of a polypeptide that has the cinnamyl alcohol dehydrogenase function and consists of the amino acid sequence of SEQ ID NO. 2 or its equivalent sequence.

9. A composition for inhibiting plant growth comprising a growth inhibitor screened by the process set forth in claim 8.

10. The composition according to claim 9, in which the inhibitor is selected from a group consisting of (1) the anti-sense nucleotide against the polynucleotide of claim 2; (2) the recombinant vector containing the anti-sense nucleotide against the polynucleotide of claim 2; and (3) the transformant of Agrobacterium tumefaciens transformed with the recombinant vector containing the anti-sense nucleotide against the polynucleotide of claim 2.


SEQUENCE LISTING (6 sequences)

SEQ ID NO: 1 - DNA, 1205 bp, Arabidopsis thaliana
acgcattcgtcatttggtccctccacttagagagaagcagacagaacataactaaaatcgagaaaaatggcaaacagtggtgaaggtaaagtggtgtgtgtaacaggagcctccggttacatcgcctcatggctcgtcaagttcctacttagccgtggctacactgttaaggcctccgtccgtgatcccagtgatccgaaaaagacacaacacttagtttcactagaaggtgcaaaggaaagacttcacttgttcaaagcagaccttttggaacaaggttctttcgactctgctattgatggttgccatggagttttccacactgcttctccattttttaatgatgccaaagacccacaggctgaacttattgatcctgcggtcaaggggacgcttaacgttttgaattcgtgcgccaaagcctcttcggttaagagggttgttgtaacctcctccatggctgccgttggttacaatggaaaaccacgcacacctgatgttaccgtcgatgaaacttggttctctgatcctgagctttgcgaggcctccaagatgtggtatgttctatccaagactttggcggaagatgcagcttggaaactcgctaaagagaaaggcttagacattgttactattaacccggctatggtgatcggtcctctcctacagccaactctgaacacgagtgctgctgctatattaaacttaatcaatggtgcaaagactttcccaaacttgagtttcggatgggttaatgtaaaagacgtagccaatgcgcacatccaaacatttgaggtcccttcagctaatgggcgttattgtttggtcgagcgtgtcgttcaccactccgagattgttaacattctacgtgagctttacccaaatctcccactacctgaaaggtgtgtggacgagaatccctacgtgccaacgtatcaagtgtccaaggataaaacgaggagccttggcatagactacatacccttgaaggttagcatcaaggagaccgtcgagtccttgaaggaaaaaggtttcgcacagttctgagaaagcatttgagccaatggatttaatccagattagataaagtatttggaagactatttcaaaaataatatttggaacatgtcaatgttctcaaggagatattagtatgttcttgtgtactttattgttgttccatcaaatgagttacttttcctttt

SEQ ID NO: 2 - protein, 326 aa, Arabidopsis thaliana
MANSGEGKVVCVTGASGYIASWLVKFLLSRGYTVKASVRDPSDPKKTQHLVSLEGAKERLHLFKADLLEQGSFDSAIDGCHGVFHTASPFFNDAKDPQAELIDPAVKGTLNVLNSCAKASSVKRVVVTSSMAAVGYNGKPRTPDVTVDETWFSDPELCEASKMWYVLSKTLAEDAAWKLAKEKGLDIVTINPAMVIGPLLEPTLNTSAAAILNLINGAKTFPNLSFGWVNVKDVANAHIQAFEVPSANGRYCLVERVVHHSEIVNILRELYPNLPLPERCVDENPYVPTYQVSKDKTRSLGIDYIPLKVSIKETVESLKEKGFAQF

SEQ ID NO: 3 - DNA, 35 bp, Artificial
  Sense Primer
aaggatccatggcaaacagtggtgaaggtaaagtg

SEQ ID NO: 4 - DNA, 35 bp, Artificial
  Antisense Primer
cgaagctttcagaactgtgcgaaacctttttcctt

SEQ ID NO: 5 - DNA, 31 bp, Artificial
  Sense Primer
gaagatctcagaactgtgcgaaacctttttc

SEQ ID NO: 6 - DNA, 29 bp, Artificial
  Antisense Primer
gctctagatggcaaacagtggtgaaggta